(19) United States
(12) Patent Application Publication
(10) Pub. No.: US 2006/0190012 A1
(43) Pub. Date: Aug. 24, 2006

(54) Method and apparatus for representing an instrument relative to a bone

(75) Inventor: Harry Freitag, Kirchzarten (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/339,339

(22) Filed: Jan. 24, 2006

(30) Foreign Application Priority Data
Jan. 29, 2005 (DE) 10 2005 004 192

(51) Int. Cl. A61B 19/00

(52) U.S. Cl. 606/130

(57) ABSTRACT

To enable the position and orientation of an instrument which machines a bone to be determined relative to the bone without the need for X-radiation, the invention proposes a method for representing an instrument relative to a bone, in which the structure of the bone in a working plane passing through the bone is determined by means of an ultrasound head which emits and receives ultrasonic radiation in the working plane, in which the three-dimensional position and orientation of the bone and of the ultrasound head are determined by navigation, and from this information the position and orientation of the structure of the bone in a system of coordinates that is fixed with respect to the bone is determined, in which the ultrasound image in this working plane is represented on a display, in which the navigated instrument is aligned in such a way relative to the bone that its working direction is located in the working plane, and in which the working region of the instrument in the working plane is copied in the correct position into the representation of the ultrasound image on the display. The invention also describes an apparatus for carrying out the method.

The present disclosure relates to the subject matter disclosed in German application No. 10 2005 004 192.2 of Jan. 29, 2005, which is incorporated herein by reference in its entirety and for all purposes.

### BACKGROUND OF THE INVENTION

The invention relates to a method for representing an instrument relative to a bone and to an apparatus for carrying out this method.

Medical operations in many cases involve machining a bone by means of an instrument; this instrument may, for example, be a drill, a saw, a milling cutter or another material-removing machining instrument, or may alternatively be a bone screw that is to be screwed into the bone or a pin to be driven into the bone or the like. In all cases, it is important for the position of this part, which is referred to in general terms below as the instrument, in the bone to be determined, in order to prevent incorrect positioning and in particular injuries caused by the instrument advancing too far within the bone. This monitoring of position and location of the instrument relative to the bone can be carried out with the aid of X-rays or similar imaging methods, but this involves this observation taking place throughout the entire operation, which can lead to very considerable levels of radiation and is also very laborious.

It is an object of the invention to provide a method which allows the position of the instrument relative to the bone to be monitored in a simple way without the need for X-radiation of this nature.

### SUMMARY OF THE INVENTION

According to the invention, this object is achieved by a method for representing an instrument relative to a bone, in which the structure of the bone in a working plane passing through the bone is determined by means of an ultrasound head which emits and receives ultrasonic radiation in the working plane, in which the three-dimensional position and orientation of the bone and of the ultrasound head are determined by navigation, and from this information the position and orientation of the structure of the bone in a system of coordinates that is fixed with respect to the bone is determined, in which the ultrasound image in this working plane is represented on a display, in which the navigated instrument is aligned in such a way relative to the bone that its working direction is located in the working plane, and in which the working region of the instrument in the working plane is copied in the correct position into the representation of the ultrasound image on the display.

Therefore, the structure of the bone and the working region of the instrument can be seen simultaneously in the correct position with respect to one another on a display of this type, the representation showing a two-dimensional image in the working plane. The working direction of the instrument is in this case, for example, the direction of advance of a saw in the sawing plane, the direction of advance of a drill, the screw-in direction of a bone screw, etc.

With the aid of the ultrasound head, it is possible to determine the bone structure by transit time measurements for the ultrasonic radiation between emission and reflection from the bone structure, and with the aid of the location and position determination of both the ultrasound head and the bone with the aid of the navigation system it is thereby possible to determine the position of the bone structure in a system of coordinates that is fixed with respect to the bone. At the same time, the instrument is disposed in such a way relative to the bone that its working direction is located in the working plane, and the instrument itself is likewise navigated, i.e. its position and location relative to the system of coordinates that is fixed with respect to the bone can be calculated at any time. This makes it possible for the instrument or the working region of the instrument to be represented in the correct position in the two-dimensional representation of the display relative to the bone structure, even though the ultrasound head can only determine the bone structure but not the instrument. The instrument is normally disposed in a region of the bone which cannot be reached by the ultrasonic radiation, and therefore its position cannot be determined by the ultrasound head. Nevertheless, a representation which also shows the position of the instrument in the space that is inaccessible to radiation correctly still appears on the display.

It is not normally a true image of the instrument which is represented on the display, but rather a schematic image, for example the region which is covered by an oscillating saw blade or the region in which a drill is producing a hole. In this context, the term working region is substantially to be understood as meaning those regions which penetrate furthest into the bone and at which a change in the bone substance takes place, for example the tip of a drill, the cutting edge of a saw or the tip of a bone screw.

One significant advantage of the method described is that the position of the bone structure relative to the system of coordinates that is fixed with respect to the bone can be determined by a single ultrasound measurement which can be carried out at the beginning of the operation, i.e. there is no need for ongoing measurement with exposure to radiation during the operation. Once the position of the bone structure in the system of coordinates that is fixed with respect to the bone has been determined, it is possible to determine the relative position of the working region of the instrument with respect to the bone structure purely by means of the navigation-assisted location and orientation determination of the bone and of the instrument.

A significant facilitation results from the fact that the plane covered by the ultrasound beams is located in the working plane of the bone and that also the instrument is disposed in such a way that the working direction is located in this working plane. As a result, it is possible to display a two-dimensional representation of this working plane which provides the operator with all the information as to the profile of the working region of the instrument in this working plane relative to the bone structure. The operator can then, for example, accurately determine the sawing depth or the screw-in depth of a bone screw without additional position determinations of the instrument, involving the use of radiation, being required.

According to a first preferred exemplary embodiment of, the invention, it is possible to provide that the instrument is manually disposed with the aid of the location and position determination by the navigation in such a way that its working direction is located in the working plane. For example, by a suitable representation on the display, it is possible to establish whether the working plane of the instrument deviates from the plane in which the ultrasound head emits its radiation and receives it again, and the instrument can then be displaced and rotated in such a way that its working plane coincides with this radiation plane.

In another preferred exemplary embodiment, this coordination of the relative position of instrument and working plane determined by the original measurement orientation of the ultrasound head is achieved with the aid of a controlled positioning device; the latter can be controlled by the navigation system via a data processing device, which determines the radiation plane of the ultrasound head and disposes the instrument in such a way that its working plane is located in this radiation plane.

It is particularly advantageous if the instrument and the ultrasound head are disposed on opposite sides of the bone, in which case the instrument is guided within the space of the bone that is free of ultrasonic radiation, since the ultrasonic radiation is reflected at the surface of the bone and therefore marks this surface as bone structure.

For certain operations, it is possible to provide that the structure of the bone is determined in various working planes in succession and the instrument is successively moved into these working planes. By way of example, it is thus possible to determine the boundary surfaces of a wedge-shaped piece of bone which is to be sawn out of the bone with the aid of a saw.

The representation method described enables the operator to interrupt the process of machining the bone by the instrument as soon as the distance between the working region of the instrument and the structure of the bone drops below a defined minimum value.

Moreover, the invention is based on the object of proposing an apparatus for representing an instrument relative to a bone.

According to the invention, this object is achieved by an apparatus having a navigation system for determining the location and orientation of a bone, an ultrasound head, which emits and receives ultrasonic radiation in a working plane, and an instrument which machines the bone, and having a data processing system, which is programmed in such a way that it represents the structure of the bone which has been determined by the ultrasound head in the working plane on a display and at the same time represents the working region of the instrument disposed with its working direction in the working plane.

Furthermore, it is possible to provide that the instrument has associated with it a positioning device, which aligns the instrument relative to the bone in such a way that its working direction is located in the working plane.

It is advantageous if the emission direction of the ultrasound head is opposite to the working direction of the instrument.

The instrument may, for example, be a saw, the sawing plane of which is located in the working plane.

In another exemplary embodiment, the instrument is a drill, the direction of advance of which is located in the working plane.

It is also possible for the instrument to be a bone screw, the screw-in direction of which is located in the working plane.

The following description of preferred embodiments of the invention, in conjunction with the drawing, serves to provide a more detailed explanation.

### BRIEF DESCRIPTION OF THE DRAWINGS:

FIG. 1: shows a schematic illustration of a bone with an ultrasound head and an oscillating saw as well as a navigation system for the ultrasound head, for the oscillating saw and the bone, a data processing system and a display for representing a working plane of the bone;

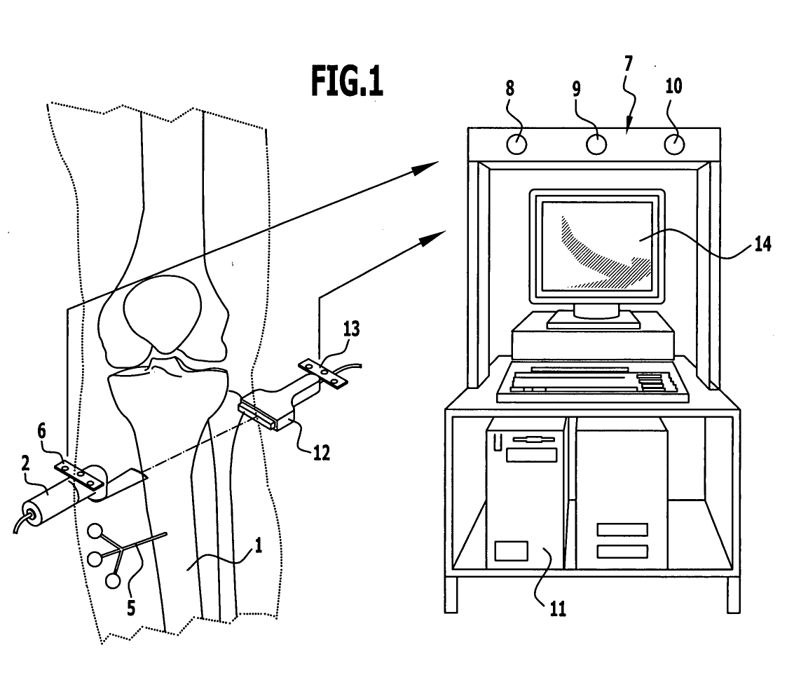

FIG. 2: shows a view of the bone together with an ultrasound head and an oscillating saw similar to FIG. 1, with two working planes that are inclined with respect to one another and define a wedge-shaped bone portion, and

FIG. 3: shows a schematic illustration of the display of the bone structure and of the working region of an instrument in the working plane, with the ultrasound head and the instrument additionally being represented in order to define the ultrasound direction and the working direction.

### DETAILED DESCRIPTION OF THE INVENTION

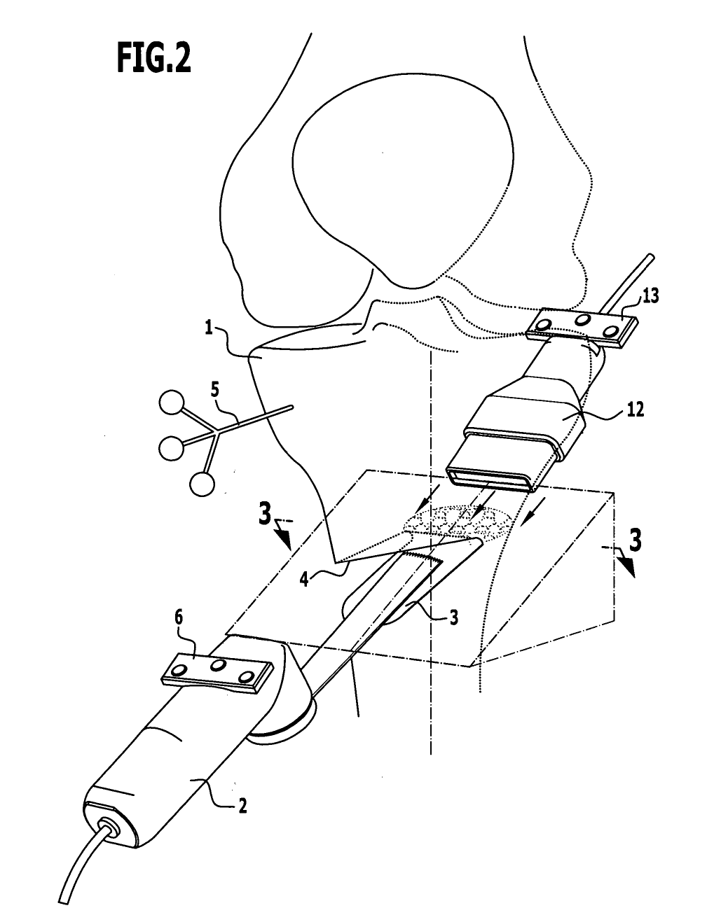

The invention is explained below based on the example of the machining of a tubular bone, in particular the tibia 1, into which two cuts 3, 4, which are inclined relative to one another, are to be introduced with the aid of an oscillating saw 2, in order thereby for a wedge-shaped piece of the bone to be cut out of it (FIG. 2). However, it will be understood that the invention is also suitable for other machining operations carried out on the bone, for example for drilling holes, for screwing in bone screws or the like.

To carry out the method, marker elements 5 and 6 are fixed to the tibia 1 and the oscillating saw 2, respectively, the three-dimensional position and orientation of which marker elements can be determined by a stationary navigation system 7. Marker elements and navigation systems of this type are known; they work, for example, with a plurality of stationary emitters 8, 9, 10 which are disposed at a distance from one another and emit infrared radiation. This infrared radiation is reflected by the marker elements 5, 6 at locations that are spatially separate from one another and is picked up by receivers that are spatially separate from one another on the navigation system 7, after which a data processing system 11 associated with the navigation system 7 can determine the three-dimensional orientation and position of the marking elements from the direction and transit times of the radiation. Of course, it is also possible to use other navigation systems to determine the position of these marker elements 5, 6, for example electromagnetic, optical, etc.

An ultrasound head 12, which, in a manner known per se, emits ultrasonic radiation in a radiation plane and receives this radiation again after it has been reflected, is used to carry out an operation procedure. The distance and position of a reflecting surface can be determined by the ultrasound head 12 from the radiation direction and the transmit time, and this takes place in the entire plane covered by the ultrasound radiation, so that the position of a reflection surface in this plane can be determined. A reflection surface of this type is formed, for example, by the outer surface of the tibia 1.

The ultrasound head 12 also has an associated marker element 13, so that the three-dimensional position of the ultrasound head 12 can also be determined by the navigation system 7.

When the ultrasound head 12 is directed onto the tibia 1, the ultrasound head 12 defines a plane in which the ultrasound radiation impinges on the tibia 1. The bone structure of the femur 1 is determined in this plane, and since both the position and orientation of the ultrasound head 12 and of the tibia 1 are known on account of the navigation system 7, it is in this way possible to determine the position of the bone structure, i.e. of the outer side of the tibia 1 in a system of coordinates that is fixed with respect to the bone, i.e. relative to the marker element 5 of the tibia 1.

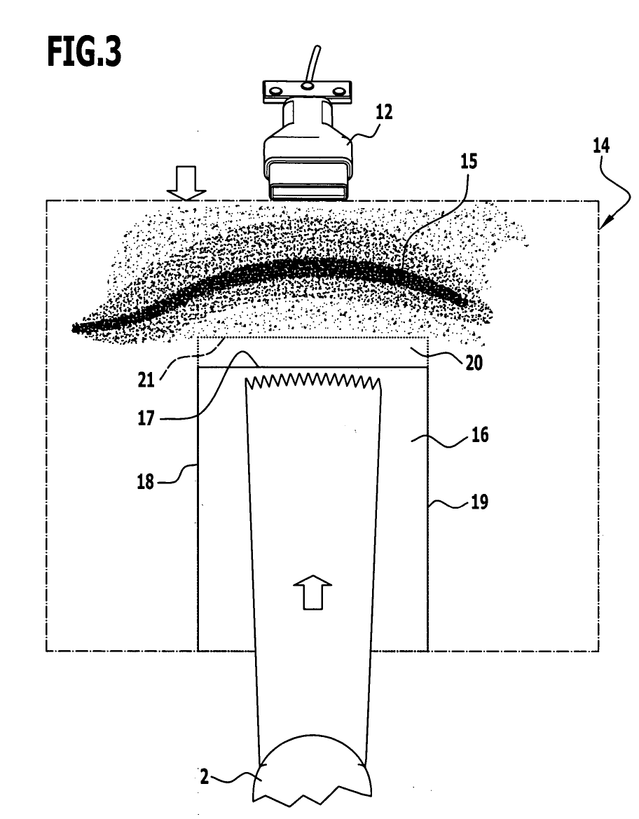

The data processing system 11 uses the data obtained in this way to generate an image of the plane covered by the ultrasonic radiation of the ultrasound head 12 (referred to below as the working plane) on a screen 14. The bone structure 15, which is an image of the outer surface of the tibia 1, on the side of the tibia impinged on by the ultrasonic radiation, can be seen in this working plane. A bone structure 15 of this type is represented in the display shown in FIG. 3, which also symbolically indicates the ultrasound head 12, in order to demonstrate the direction in which the ultrasonic radiation of the ultrasound head 12 is directed onto the tibia 1. In reality, of course, an ultrasound head 12 of this type is not represented on the display and is also not required.

The working plane of the ultrasound head is selected in such a way that it corresponds to the working plane of the instrument, i.e. that this working plane coincides with the plane in which a sawing cut is to be carried out. This can be predefined by suitable planning, and then to record the bone structure the ultrasound head 12 is oriented in such a way relative to the tibia 1 that the plane covered by its radiation coincides with this desired cutting plane. The bone structure 15 is determined with this orientation of the ultrasound head, and this plane is then represented on the screen 14.

In a following step, the oscillating saw 2 is displaced and rotated with the aid of the navigation system in such a way that its working plane, i.e. for example the cutting plane of the oscillating saw, coincides with the plane covered by the ultrasonic radiation of the ultrasound head 12. This can be done by hand or by means of a positioning device which holds the oscillating saw 2 and is suitably controlled by the navigation system 7 via the data processing system 11. Therefore, the working plane of the instrument is in an accurately known plane relative to the tibia 1 and this corresponds precisely to the plane which is represented on the screen 14. Since the oscillating saw 2 has been navigated, the data processing system 11 also has data which represent the precise position and orientation of the oscillating saw 2, which means that it is possible, together with stored geometric data for the oscillating saw 2, to represent the working area of the saw on the screen 14, specifically in such a way that it is copied into the representation on the screen 14 in the correct position with respect to the bone structure 15. The working area may, for example, be the position of the cutting line and/or the width of the oscillation movement of the saw blade; in FIG. 3, this working area is represented by a rectangle 16, in the area of which a sawing cut is disposed when the oscillating saw 2 is working. A boundary line 17 of this rectangle 16 indicates the depth of penetration of the oscillating saw 2, while the lateral boundary lines 18, 19 indicate the width of the sawing cut which is produced.

In this way, the operator can accurately follow on the screen 14 how far the sawing cut has progressed, and in particular that the penetration depth has not exceeded a defined maximum value at which the saw has approached the bone structure 15 as far as a minimum value.

A cutting region 20 has also been copied into the representation in FIG. 3 by means of dotted lines; this cutting area corresponds to the planned sawing cut, i.e. it shows how far away the actual sawing cut is from the desired sawing cut. When the front boundary line 17, which corresponds to the actual sawing depth, reaches the parallel dotted line 21 of the predetermined sawing structure, the operator must not advance the oscillating saw 2 any further, but rather he is aware that the actual sawing cut corresponds to the planned sawing cut.

Purely for illustration, FIG. 3 additionally shows the oscillating saw 2, in order to demonstrate how it works and how it is advanced in the direction of the bone structure 15. In accordance with the preferred actual formation, in the illustration in FIG. 3 the ultrasound head 12 and oscillating saw 2 are positioned opposite one another, i.e. the introduction of the oscillating saw 2 into the tibia 1 takes place in the direction of the ultrasound head 12 disposed opposite it.

Of course, there is no need for the ultrasound head 12 to remain in this position during the sawing operation and to continue to carry out measurements, but rather it is sufficient for the bone structure 15 to have its orientation and position on the system of coordinates that is fixed with respect to the tibia, and therefore relative to the marker element 5, to be determined once at the start of the procedure.

To cut out a wedge-shaped bone element, as indicated in the exemplary embodiment shown in FIG. 2, it is possible for a corresponding procedure to be carried out for two different working planes in succession, i.e. in accordance with each of the two cutting planes, which are inclined with respect to one another, in first instance a location determination of the bone structure in the tibia-side system of coordinates is carried out by orientation of the ultrasound head in these planes, and then this plane is represented on the screen 14, and for machining purposes the oscillating saw 2 is then oriented into this working plane and advanced into the tibia down to the desired working depth. The same procedure then takes place for the second plane. This ensures that the tibia 1 cannot be completely severed, the operator can track the progress and depth of the actual sawing cut at any time on the screen 14, even though the oscillating saw 2 and its saw blade are located in a region of the tibia 1 which is not reached by the ultrasonic radiation from the ultrasound head 12.

1. Method for representing an instrument relative to a bone, in which the structure of the bone in a working plane passing through the bone is determined by means of an ultrasound head which emits and receives ultrasonic radiation in the working plane, in which the three-dimensional position and orientation of the bone and of the ultrasound head are determined by navigation, and from this information the position and orientation of the structure of the bone in a system of coordinates that is fixed with respect to the bone is determined, in which the ultrasound image in this working plane is represented on a display, in which the navigated instrument is aligned in such a way relative to the bone that its working direction is located in the working plane, and in which the working region of the instrument in the working plane is copied in the correct position into the representation of the ultrasound image on the display.

2. Method according to claim 1, wherein the instrument is manually disposed with the aid of the location and position determination by the navigation in such a way that its working direction is located in the working plane.

3. Method according to claim 1, wherein the instrument is disposed by means of a positioning device controlled by the navigation system in such a way that its working direction is located in the working plane.

4. Method according to claim 1, wherein the instrument and the ultrasound head are disposed on opposite sides of the bone.

5. Method according to claim 1, wherein the structure of the bone is determined in various working planes in succession, and the instrument is successively moved into these working planes.

6. Method according to claim 1, wherein a bone machining process by the instrument is interrupted as soon as the distance between the working region of the instrument and the structure of the bone drops below a defined minimum value.

7. Apparatus for representing an instrument relative to a bone, having a navigation system for determining the location and orientation of the bone, an ultrasound head, which emits and receives ultrasonic radiation in a working plane, and an instrument which machines the bone, and having a data processing system, which is programmed in such a way that it represents the structure of the bone which has been determined by the ultrasound head in the working plane on a display and at the same time represents the working region of the instrument disposed with its working direction in the working plane.

8. Apparatus according to claim 7, wherein the instrument has associated with it a positioning device, which aligns the instrument relative to the bone in such a way that its working direction is located in the working plane.

9. Apparatus according to claim 7, wherein the emission direction of the ultrasound head is opposite to the working direction of the instrument.

10. Apparatus according to claim 8, wherein the emission direction of the ultrasound head is opposite to the working direction of the instrument.

11. Apparatus according to claim 7, wherein the instrument is a saw, the sawing plane of which is located in the working plane.

12. Apparatus according to claim 8, wherein the instrument is a saw, the sawing plane of which is located in the working plane.

13. Apparatus according to claim 7, wherein the instrument is a drill, the direction of advance of which is located in the working plane.

14. Apparatus according to claim 7, wherein the instrument is a bone screw, the screw-in direction of which is located in the working plane.
